(19) United States
(12) Patent Application Publication
(10) Pub. No.: US 2009/0076132 A1
(43) Pub. Date: Mar. 19, 2009

(54) ANTIVIRAL COMPOSITIONS AND METHODS OF TREATMENT

(76) Inventor: Gary Pekoe, Virginia Beach, VA (US)

Correspondence Address: WILLIAMS MULLEN, 222 CENTRAL PARK AVENUE, SUITE 1700, VIRGINIA BEACH, VA, 23462, US

(21) Appl. No.: 12/234,342

(22) Filed: Sep. 19, 2008

Related U.S. Application Data
(63) Continuation-in-part of application No. 12/022,627, filed on Jan. 30, 2008.
Application No. 12/022,627 is a continuation-in-part of application No. 11/078,987, filed on Mar. 11, 2005, now abandoned.

(51) Int. Cl. A61K 31/353; A61P 31/20

(52) U.S. Cl. 514/456

(57) ABSTRACT

A composition and method of treatment of viruses in the Poxviridae family of infections and their associated symptoms using a tree extract out of which the majority (>90%) of dry mass are hydrolyzing flavonoids. Other extract components include taspine, dihydrobenzofuran lignan, phenols, steroids; and diterpenoids. The composition, flavokine, has been previously shown to treat various viral infections and their associated symptoms such as Molluscum Contagiosum. It has also been shown to treat various bacterial infections such as the Staphylococci, Streptococci, and Enterococcus faecalis family of infections and their associated symptoms. In the present invention, flavokine, is formulated and administered to treat cowpox and vaccinia.

### RELATED APPLICATION DATA

This application is a continuation-in-part of application Ser. No. 12/022,627 filed Jan. 30, 2008, which is a continuation-in-part of application Ser. No. 11/078,987 filed Mar. 11, 2005. Each of these documents is incorporated by reference in their entirety.

### FIELD OF THE INVENTION

The invention relates to a composition and method of treatment for Poxviruses (members of the family Poxviridae) using an extract from the lechleri Croton tree.

### BACKGROUND OF THE INVENTION

Viruses are a problem across the world, causing many of the diseases that afflict individuals today. Some of the most terrible and difficult to manage viruses belong to the Poxviridae family. Poxviruses are a family of viruses that can infect both humans and animals. Three particularly troublesome Poxviruses are: smallpox, cowpox and molluscum contagiosum. Smallpox and cowpox are species of the Orthopoxvirus genus. Molluscum contagiosum is a species from the Molluscipoxvirus genus. Both genera are part of the Poxviridae family.

Smallpox is also known by the Latin names Variola or Variola vera, which is a derivative of the Latin varius, meaning spotted, or varus, meaning “pimple.” Smallpox has produced significant death in the human population in the past. During the 20th century, it is estimated that smallpox was responsible for 300-500 million deaths. After successful vaccination campaigns throughout the 19th and 20th centuries, the World Health Organization certified the eradication of smallpox in 1979. It is now confined to licensed laboratory analysis. It is also suspected to be a potential bioterrorism agent. Smallpox virus preferentially attacks skin cells, causing the characteristic pimples (called macules) associated with the disease. A rash develops on the skin 24 to 48 hours after lesions on the mucous membranes appear. Typically the macules first appear on the forehead, then rapidly spread to the whole face, proximal portions of extremities, the trunk, and lastly to distal portions of extremities. The process can occur in as little as 24 to 36 hours, after which no new lesions appear.

According to the CDC the vaccinia virus (“VV”) is the “live virus” used in the smallpox vaccine. It too is a “pox”-type virus related to smallpox, but milder. When given to humans as a vaccine, it helps the body to develop immunity to smallpox. The smallpox vaccine does not contain the smallpox virus and it cannot cause smallpox although sometimes a person getting a live vaccine can experiences mild symptoms associated with the virus such as rash, fever, and head and body aches.

Smallpox vaccination within three days of exposure will prevent or significantly lessen the severity of smallpox symptoms in the vast majority of people. According to some sources, vaccination 4 to 7 days after exposure likely offers some protection from or modifies the severity of the disease. Other than vaccination, treatment of smallpox is primarily supportive, such as wound care and infection control, fluid therapy, and possible ventilator assistance. No drug is currently approved for the treatment of smallpox. Laboratory tests, including those conducted by the National Institute of Allergy and Infectious Diseases (“NIAID”) are often directed against vaccinia as an indication of activity against smallpox.

Cowpox, also known as Catpox, is a disease of the skin that is caused by a virus known as the cowpox virus (“CV”). Cowpox is also related to vaccinia and got its name from cow maids touching the udders of infected cows. The cowpox virus was actually used to perform the first successful vaccination against smallpox. Although human cases of cowpox today are very rare, and most often contracted from domestic cats, a large animal population is affected by cowpox. When animals are infected with cowpox, it can have a large impact on the use of the livestock population. Symptoms of infection with CV in humans are localized, pustular lesions generally found on the hands and limited to the site of introduction. The incubation period is nine to ten days.

Another viral relative to smallpox is molluscum contagiosum (MC), which like smallpox, infects only humans. However, unlike smallpox, molluscum infection is benign. MC is a viral infection of the skin or occasionally of the mucous membranes. MC affects any area of the skin but is most common on the body, arms, and legs. It is spread through direct contact or shared articles of clothing. In adults, MC infections are often sexually transmitted and usually affect the genitals, lower abdomen, buttocks, and inner thighs. In rare cases, MC infections are also found on the lips, mouth, and eyelids. The time from infection to the appearance of lesions ranges from 1 week to 6 months, with an average incubation period of 6 weeks. Diagnosis is made on the clinical appearance; the virus cannot routinely be cultured. MC is, however, a self-limiting virus. It also resides primarily in pediatric and AIDS patients. It is thought to be responsible for 5% of all skin diseases affecting children and to infect 20% of AIDS patients.

The composition and method of treatment described herein has been shown previously to have activity in treating MC and its associated symptoms. It is now demonstrated herein to also have activity against members of the family of Poxviruses including VV and CV.

### SUMMARY OF THE INVENTION

Flavonoids are ubiquitous dietary chemicals possessing antioxidant properties. Literature strongly supports that flavonoids have profound effects on the function of immune and inflammatory cells by various means such as but not limited to secretory processes, mitogenesis, cell-cell interactions, and likely, gene expression of cytokines. Studies have also shown that some flavonoids can be effective against viruses. Some flavonoids affect the intracellular replication of viruses. Others inhibit the infectious properties of the viruses. Some flavonoids have shown both anti-replicative and anti-infectious activity. In sum, while the exact mechanism of individual flavonoids may vary, studies indicate that flavonoids affect the motion and function of viruses. That is flavonoids affect various kinetic activity of viruses.

The present invention provides a composition and method of treatment for VV and CV family of infections and their associated symptoms using an extract from the lechleri Croton tree out of which the majority (>90%) of dry mass are hydrolyzing flavonoids. Other extract components include taspine, dihydrobenzofuran lignan, phenols, steroids; and diterpenoids. Based on the majority presence of flavonoids and their varying kinetic effects on viruses, the tested extract is hereafter referred to as “flavokine” for ease of reference. Thus the term “flavokine” should be construed as referring to substantially all of the metabolites or crotonoids of an extract of the lechleri Croton tree.

Flavokine has been previously shown to treat various viral infections and their associated symptoms such as MC. It has also been shown to treat various bacterial infections such as the Staphylococci, Streptococci, and Enterococcus faecalis family of infections and their associated symptoms.

Of course, effectiveness against a bacterial strain provides no indication of activity against a viral strain. Neither does activity against one viral strain indicate effectiveness against another viral strain. Now there is surprising support that flavokine is effective against various Poxviruses of the Orthopoxvirus genus including, vaccinia and cowpox.

### DETAIL DESCRIPTION OF INVENTION

Flavokine was tested against the following viral species:

| | |
|---|---|
| Cowpox | (CV) |
| Vaccinia | (VV) |

Tests were conducted by the National Institute of Allergy and Infectious Diseases (“NIAID”) and the Antimicrobial Acquisition and Coordinating Facility (“AACF”). The results show flavokine to have activity against VV and CV.

The experimental approach of NIAID's AACF for determining antiviral efficacy and toxicity of antiviral agents is based on the following: 1) an inexpensive, rapid assay such as a cytopathic effect inhibition assay (CPE-inhibition assay) that is semi-automated is used initially to screen out the negatives; 2) all screening assays are conducted in low-passaged human cells; 3) each assay system contains a positive control (CDV) and a negative control (ACV); 4) efficacy is demonstrated by at least two different assay systems that detect functional biologic activity; and 5) toxicity is determined using both resting and proliferating human fibroblast cells.

Compounds were initially screened for activity using the CPE assay in human foreskin fibroblast (HFF) cells. The screening assay systems utilized were selected to show specific inhibition of a biologic function, i.e., cytopathic effect in susceptible human cells. In the CPE-inhibition assay, drug is added 1 hr prior to infection so the assay system will have maximum sensitivity and detect inhibitors of early replicative steps such as adsorption or penetration as well as later events. To rule out non-specific inhibition of virus binding to cells all compounds that show reasonable activity in the CPE assay are confirmed using a classical plaque reduction assay in which the drug is added 1 hr after infection. These assay systems also can be manipulated by increasing the pre-treatment time in order to demonstrate antiviral activity with oligodeoxynucleotides and/or peptides. By delaying the time of addition of drug after infection, information regarding which step in the virus life cycle is inhibited (i.e., early vs. late functions) can be gained. It is also important to determine the toxicity of new compounds on dividing cells at a very early stage of testing. Past research indicates that a cell proliferation assay using HFF cells is a very sensitive assay for detecting drug toxicity to dividing cells and the drug concentration that inhibits cell growth by 50% (IC₅₀). HFF cells are the most sensitive and predictive of toxicity for bone marrow cells, thus they are the preferred cell type to be used in these assays.

In all the assays used for primary screening, a minimum of six drug concentrations was normally used covering a range of 100 μg/ml to 0.03 μg/ml, in 5-fold increments to obtain good dose response curves. From these data, the dose that inhibited viral replication by 50% (effective concentration 50; EC₅₀) was calculated. The same drug concentrations used to determine efficacy were also used on uninfected cells in each assay to determine toxicity of flavokine. The drug concentration that was cytotoxic to cells as determined by their failure to take up a vital stain, neutral red, (cytotoxic concentration 50; CC₅₀) was determined as above. To determine if flavokine has sufficient antiviral activity that exceeds its level of toxicity, a selectivity index (SI) was calculated according to CC₅₀/EC₅₀. This index, also referred to as a therapeutic index, was used to determine if a compound warrants further study.

Results were reported by an AACF Compound Screening Program Medicinal Chemist. The concentrations in the reported CPE assay were 50, 10, 2, 0.4, 0.08 and 0.016%. The reported EC₅₀ values for both VV and CV was <0.016%. The optical density readings were higher for all concentrations tested for this compound compared to the average cell control readings in the neutral red uptake and crystal violet cytotoxicity assays, as well as the VV and CV virus controls. The CC₅₀ for both VV and CV was >50% and the SI (selectivity index) was >3125 for each (see Table 1 and 2 below).

TABLE 1
CPE Assay: Results of flavokine against CV and VV using HFF Cells.

| | | Cell | | Drug | | | | | | Ctl | Ctl |
|---|---|---|---|---|---|---|---|---|---|---|---|
| ARB # | Virus | Line | Trial | Unit | EC50 | EC90 | CC50 | SI | Control | EC50 | EC90 |
| 05- | Cowpox | HFF | 1 | % soln | <0.016 | <0.016 | >50 | >3125 | CDV | 7.2 | 14.4 |
| 104305 | | Cells | | | | | | | | | |
| | Vaccinia | HFF | 1 | % soln | <0.016 | <0.016 | >50 | >3125 | CDV | 2.8 | 4.0 |
| | | Cells | | | | | | | | | |

TABLE 2
Neutral red uptake results for the toxicity of flavokine in uninfected HFF cells.

| | Cmpd | | | | Drug | | | Control |
|---|---|---|---|---|---|---|---|---|
| ARB # | Name | Assay | Cell Line | Trial | Unit | CC50 | Control | CC50 |
| 05- | PoxiDerm | Toxicity - | Stationary | 1 | % soln | >50 | CDV | >100 |
| 104305 | | Neutral | HFF | | | | | |
| | | Red | Cells | | | | | |
| | | Uptake | | | | | | |

Based on these results, flavokine appears to be very active against both cowpox and vaccinia viruses. Low doses of flavokine inhibit viral replication and have not been found to be toxic to uninfected cells. The effective concentration of flavokine was much lower than that of the control, indicating that this product is very effective as a treatment for cowpox and vaccinia virus.

This supports expanded use of flavokine against beyond Molluscum Contagiosum in the family of Poxviruses. Consistent with the literature, one of the mechanisms of action of flavokine appears to be its ability to inhibit the activity or kinetics of the Poxvirus, which accounts, at least partly, for the clinical results. Future tests are recommended to examine the effectiveness of flavokine against other similar viruses, as well as other, non Pox-type viruses.

Thus it will be appreciated that the composition in accord with the invention, for use in treating the Poxviridae family of infections including VV and CV and their associated symptoms, is comprised of flavokine or one or more individual components of flavokine.

Flavokine may be obtained from any of a number of tree species within the genus Croton (family Euphorbiaceae) that grow in Central and South America such as but not limited to the Amazon region of Peru, Ecuador, Brazil, Dominican Republic, Mexico and Colombia. Other Croton species may also be used as a source for flavokine. Other Croton species include Croton salutaris, Croton gossypifolius, Croten palanostima, Croton erythrochilus, Croton urucurana, Croton xalapensis and Croton draconoides. Flavokine may be alternately derived from one tree, multiple trees of the same species, or from multiple trees of different species.

Flavokine is a composition comprising compounds such as but not limited to Cyanidole (flavonolmonomers): (+)-gallocatechin, (+)-catechin, (−)-epicatechin, (−)-epigallocatechin, proanthocyanidin(proanthocyanidindimerB-4, proanthocyanidintrimer, proanthocyanidintetramer, proanthocyanidinheptamer), cedrucine, Daucosterol, Dihydrobenzofuran, Dimethylcedrusine, Isoboldine, korberinA&B, magnoflorine, norisoboldine, procyanidins, resin, tannin, hardwickiicacid, bincatriole, crolechinole, crolechinicacid, coberineA, coberineB, taspine, dihyhrobenzofuranlignans:, -3x,4-O-dimethylocedrusine, -4-O-methylocedrusine, 1,3,5-trimethoxybenzene, 2,4,6-trimethoxyphenol, 4-hydroxyphenethylalcohol, beta-sitosterol, beta-sitosterol-beta-D-, glucopyranoside, beta-Pinene, Betaine, Borneol, Calamerene, Camphene, Cuparophenol, D-Limonen, Dipentene, EO, Eugenol, Euparophenol, alpha-calacorene, alpha-copaene, alpha-pinene, alpha-thujone, beta-Caryophyllene, beta-Elemene, Gamrna-Terpinene, Gamma-Terpinole, Lignin, Linalool, Methylthymol, Myrcene, Para-cymene(p-cymene), Pectic-acid, Terpinene-4-ol, Vanillin, piridona, aporfina, quinoleina, and the SP-303 (including:alkaloids, proanthocyanidins (antioxidants), terpines, diterpenes, phenols, tannins, and lignans) simplephenols, phytosterols, and biologically active alkaloids.

Another analysis of flavokine indicates the presence of the following compounds;

| Compound | Type |
|---|---|
| BENZENE, 1-3-5 TRIMETHOXY | BENZENOID |
| BENZOFURAN-5-YL,2-3-DIHYDRO:2- | LIGNAN |
| (3-4-DIMETHOXY-PHENYL): 7- | |
| METHOXY-3-METHOXY-CARBONYL- | |
| PROPAN-1-OIC ACID METHYL ESTER | |
| BENZOFURAN-5-YL,2-3-DIHYDRO: 2- | LIGNAN |
| (3-4-DIMETHOXY-PHENYL): 7- | |
| METHOXY-3-METHOXY-CARBONYL- | |
| PROPEN-1-OIC-ACID METHYL ESTER | |
| BENZOFURAN-5-YL, 2-3-DIHYDRO: 2- | LIGNAN |
| (4-HYDROXY-3-METHOXY-PHENYL)- | |
| 7-METHOXY-3-METHOXY- | |
| CARBONYL-PROPEN-1-OIC ACID | |
| METHYL ESTER | |
| BENZYL ALCOHOL, 3-4-DIMETHOXY | BENZENOID |
| BINCATRIOL | DITERPENE |
| BOLDINE, ISO | ISOQUINOLINE |
| | ALKALOID |
| BOLDINE, ISO: NOR: | ISOQUINOLINE |
| | ALKALOID |
| CATECHIN(4-ALPHA-8)- | FLAVONOID |
| GALLOCATECHIN(4-ALPHA-6)- | |
| GALLOCATECHIN | |
| CATECHIN(4-ALPHA-8)- | FLAVONOID |
| GALLOCATECHIN(4-ALPHA-8)- | |
| GALLOCATECHIN, (+): | |
| CATECHIN, (+): | FLAVONOID |
| CATECHIN, EPI: (−): | FLAVONOID |
| CEDRUSIN, 3′-4-0-DIMETHYL: | LIGNAN |
| CEDRUSIN, 3′-4-0-DIMETHYL: (DL): | LIGNAN |
| CEDRUSIN, 4-0-METHYL: | LIGNAN |
| CROLECHINIC ACID | DITERPENE |
| CROLECHINOL | DITERPENE |
| DAUSCOSTEROL | STEROID |
| GALLOCATECHIN(4-ALPHA-6)-EPI- | FLAVONOID |
| GALLOCATECHIN | |
| GALLOCATECHIN(4-ALPHA-8)-EPI- | FLAVONOID |
| CATECHIN | |
| GALLOCATECHIN(4-ALPHA-8)- | FLAVONOID |
| GALLOCATECHIN(4-ALPHA-8)-EPI- | |
| GALLOCATECHIN | |
| GALLOCATECHIN(4-ALPHA-8)- | FLAVONOID |
| GALLOCATECHIN-(4-ALPHA-8)-EPI- | |
| GALLOCATECHIN | |
| GALLOCATECHIN, (+): | FLAVONOID |
| GALLOCATECHIN, EPI: (−): | FLAVONOID |
| GLAUCINE | ISOQUINOLINE |
| | ALKALOID |
| HARDWICKIIC ACID | DITERPENE |
| KORBERIN A | DITERPENE |
| KORBERIN B | DITERPENE |
| MAGNOFLORINE | ISOQUINOLINE |
| | ALKALOID |
| PHENETHYL ALCOHOL, 4- | BENZENOID |
| HYDROXY: | |
| PHENETHYL ALCOHOL, 4- | BENZENOID |
| HYDROXY: ACETATE | |
| PHENOL, 2-4-6-TRIMETHOXY: | BENZENOID |
| PHENOL, 3-4-DIMETHOXY: | BENZENOID |
| PROCYANIDIN B-1 | FLAVONOID |
| PROCYANIDIN B-2 | FLAVONOID |
| PROCYANIDIN B-4 | FLAVONOID |
| SINOACUTINE | ISOQUINOLINE |
| | ALKALOID |
| SITOSTENONE, BETA: | STEROID |
| SITOSTEROL, BETA: | STEROID |
| SP-303 | FLAVONOID |
| TASPINE | ALKALOID |
| THALIPORPHINE | ISOQUINOLINE |
| | ALKALOID |

In one embodiment, one or more of the flavokine components through artificial manufacture may be used in the place of a naturally-obtained flavokine components and composition, wherein the artificial components and compositions approximate the activity or have substantially the same activity of the natural components and composition.

In one embodiment, flavokine is combined with a pharmaceutically acceptable diluent, adjuvant, excipient, or carrier, to facilitate and/or improve administration to a subject. Pharmaceutical formulation chemistry to create biologically active compounds and analogues can be used directly to practice materials and methods of the invention. This is a well developed art, and exemplary formulation materials and methods are discussed below.

In support of one embodiment, beta-alanyl-taurine also known as carbobenzoxy beta-alanyl-taurine is a low molecular weight benzyl sulfonic acid that promotes an effective response of the immune system. Beta-alanyl-taurine is a chemical combination of the nutrient taurine and a derivative of the amino acid alanine. Beta-alanyl-taurine has been evaluated in homeopathic provings. Preparation of beta-alanyl-taurine may be in accordance with procedures of the Homoeopathic Pharmacopoeia off the United States (HPUS). In one embodiment, beta-alanyl-taurine is used in combination with flavokine.

The preparations of beta-alanyl-taurine comprise one or more potencies of beta-alanyl-taurine at a concentration, or homeopathic potency, of less than about 10⁻⁶ molar; and in another embodiment between about 10⁻⁶ and 10^{-100,000} molar. Homeopathic preparations of the present invention are defined as comprising beta-alanyl-taurine if the preparation is derived from or originated from a preparation comprising a measurable quantity beta-alanyl-taurine.

An aspect of this embodiment is a composition comprising flavokine in combination with beta-alanyl-taurine wherein beta-alanyl-taurine has a homeopathic potency selected from the group of dilutions consisting of 2×, 3×, 4×, 5×, 6×, 7×, 8×, 9×, 10×, 11×, 12×, and so on to 24× HPUS. A variety of methods of attenuation (dilution) of beta-alanyl-taurine may be used including but not limited to the Hannemannian or Korssakovian methods as disclosed, for example, in Pub. No.: US 2004/0019107, which is hereby incorporated by reference.

In support of one embodiment, zinc functions as an antioxidant within the human body. Zinc is capable of protecting proteins and enzymes against free radical attack, or oxidation. Free radicals are unstable, reactive molecules that damage the normal functions of substances, such as cells, via rapid reactions. Zinc acts as an antioxidant to protect specific regions of enzymes from free radical attack. Through this action, zinc preserves an enzymes activity and stability. Therefore, in one embodiment, zinc is added to the composition. In another embodiment, zinc gluconate, or zinc sulfate is used in combination with flavokine wherein the zinc is within a range of about 10-100 mMol. In another embodiment, zinc gluconate, or zinc sulfate is used within a range of about 25-75 mMol.

In support of one embodiment, citrus extract, such as but not limited to orange, lemon, lime, grapefruit, and combinations thereof may be used. As used herein the term “citrus extract” refers to a solution or preparation containing the active principles of a plant, tree, fruit or the like and can be made from any or all parts of the plant, tree, fruit, or the like such as but not limited to sap, bark, roots, stems, fruit and/or leaves. Specifically, citrus extracts also function as antioxidants by reducing the amount of oxidative stress experienced in the body or individual cell. The function of citrus extract is similar to that of zinc. Therefore, in another embodiment, citrus extract is used in combination with flavokine wherein the citrus extract is added to the composition in a range of 0.5-5% W/V.

In another embodiment, zinc and citrus extract may both added to the composition within a range of about 10-100 mMol and 0.5-5% w/v respectively.

The phrase “pharmaceutically” or “pharmacologically acceptable” refers to molecular entities and compositions that do not produce adverse, allergic, or other untoward reactions when administered to a subject, e.g., topically, transdermally, parenterally, by inhalation spray, vaginally, rectally, by eye drop, or by intracranial injection. The term parenteral as used herein includes subcutaneous injections, intravenous, intramuscular, intracisternal injection, or infusion techniques. Administration by intravenous, intradermal, intramuscular, intramammary, intraperitoneal, intrathecal, retrobulbar, intrapulmonary injection and/or surgical imtreeation at a particular site is contemplated as well. Generally, this will also entail preparing compositions that are essentially free of pyrogens, as well as other impurities that could be harmful to humans or animals. The term “pharmaceutically acceptable carrier” includes any and all solvents, dispersion media, coatings, antiviral and antifungal agents, isotonic and absorption delaying agents and the like. The use of such media and agents for pharmaceutically active substances is known in the art. Topical carriers include creams, salves, foams, lotions, collagen preparations, gels, and ointments.

Flavokine may include acid or base components. As used herein, the term “component” is intended to include those naturally occurring compounds and molecules identified in flavokine as well as those formulated such as but not limited to pharmaceutically acceptable salts, esters, and combinations thereof.

For example, when an acidic substituent, such as —COOH, is present, the ammonium, sodium, potassium, calcium and like salts, are contemplated as possible embodiments for administration to a biological host. When a basic group such as amino or a basic heteroaryl radical, such as pyridyl is present, then an acidic salt, such as hydrochloride, hydrobromide, acetate, maleate, palmoate, phosphate, methanesulfonate, p-toluenesulfonate, and the like, is contemplated as a possible form for administration to a biological host.

Similarly, where an acid group is present, then pharmaceutically acceptable esters of the compound e.g., methyl, tert-butyl, pivaloyloxymethyl, succinyl, and the like are contemplated as possible forms of the compounds, such esters being known in the art for modifying solubility and/or hydrolysis characteristics for use as sustained release or prodrug formulations. In addition, some components may form solvates with water or common organic solvents. Such solvates are contemplated as part of the present invention as well.

Aqueous suspensions may contain flavokine or active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending flavokine or components of it in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Dispersible powders and granules suitable for preparation of an aqueous suspension by the addition of water provide the active composition admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

One embodiment may be formulated as a dispersable powder for dusting the skin, hair, fur, or feathers of humans or animals. One embodiment may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. Emulsions may also contain sweetening and flavoring agents and scent enhancers.

One embodiment may also be in the form of suppositories for rectal administration. These compositions can be prepared for example by mixing flavokine and/or its components with a suitable non-irritating excipient that is solid at ordinary temperatures but liquid at the rectal temperature and will therefore melt in the rectum to release the drug. Such materials may include cocoa butter and polyethylene glycols.

Therapeutic formulations may be prepared for storage by mixing the flavokine composition having the desired degree of purity with optional physiologically pharmaceutically-acceptable carriers, excipients, or stabilizers (Remington's Pharmaceutical Sciences, 18th edition, A. R. Gennaro, ed., Mack Publishing Company (1990)) in the form of a lyophilized cake or an aqueous solution. Acceptable carriers, excipients or stabilizers are nontoxic to recipients and may be inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

One embodiment to be used for in vivo administration may be sterile. This is readily accomplished by filtration through sterile filtration membranes, prior to or following lyophilization and reconstitution. One embodiment for parenteral administration will be ordinarily stored in lyophilized form or in solution.

One embodiment may be placed into a container having a sterile access port, for example, an intravenous solution bag or vial having a stopper pierceable by a hypodermic injection needle. The route of administration of the composition is in accord with known methods, e.g. topical, or by sustained release systems or imtreeation device.

Suitable examples of sustained-release preparations include semipermeable polymer matrices in the form of shaped articles, e.g. films, or microcapsules. Sustained release matrices include polyesters, hydrogels, polylactides (U.S. Pat. No. 3,773,919, EP 58,481), copolymers of L-glutamic acid and gamma ethyl-L-glutamate (Sidman, et al., Biopolymers, 22: 547-556 (1983)), poly (2-hydroxyethyl-methacrylate) (Langer, et al., J. Biomed. Mater. Res., 15:167-277 (1981) and Langer, Chem. Tech., 12:98-105 (1982)), ethylene vinyl acetate (Langer, et al., supra) or poly-D(−)-3-hydroxybutyric acid (EP 133,988). Sustained-release compositions also may include liposomes, which can be prepared by any of several methods known in the art (e.g., DE 3,218,121; Epstein, et al., Proc. Natl. Acad. Sci. USA, 82:3688-3692 (1985); Hwang, et al., Proc. Natl. Acad. Sci. USA, 77:4030-4034 (1980); EP 52,322; EP 36,676; EP 88,046; EP 143,949).

In one embodiment, flavokine is a topical composition. In one embodiment, the topical composition is formulated as a cream, a gel, an emollient, a salve, a liquid spray, an aerosol, or an impregnated bandage. In another embodiment, the topical formulation comprises a compound to improve the fragrance of the composition, including but not limited to orange extract or mint extract. In other embodiments, the composition is stored at room temperature in a product bottle, lid firmly closed, for up to one year.

### Dose and Dosing

Undiluted flavokine has proven empirically to be very safe and well tolerated in humans whether taken orally or used topically. Flavokine has been used orally by South American natives for the treatment of diarrhea, nausea, vomiting and other stomach ailments. It is has also been taken internally as a tonic, either diluted in water or brewed as a tea. It is also administered to domestic animals that exhibit gastrointestinal symptoms such as but not limited to vomiting, GI distress. Among other things, empirical data attests to the safety of the material, as untoward side effects have not been attributed to its ingestion.

Thus the frequency of application or administration to a subject can be adjusted upwardly to achieve a desired therapeutic effect. Subjects experiencing side effects should reduce dosage or discontinue use. Depending on the route of administration, a suitable dose may be calculated according to body weight, body surface areas or organ size. Further refinement of the calculations to determine the appropriate treatment dose is routinely made as part of any medical treatment regimen, in view of the pharmacokinetic data observed in animals and/or human clinical trials. Dosage consideration may also be guided by pharmaceutical references, see, e.g., Physician's Desk Reference (Montvale, N.J.), which is incorporated by reference in its entirety. Dosing in humans may also be extrapolated from animal dosages, toxicity studies, and pharmacokinetics, according to standard pharmacological methodologies.

In one embodiment, flavokine is applied via dropper onto viral-associated lesions and allowed to dry, or rubbed in gently. In another embodiment, flavokine is formulated into a cream or gel formulation and is applied on the affected area as a thin drop, and rubbed in gently. In one embodiment, a topical formulation is applied once a day up to once every 15 minutes to the affected areas.

The therapeutic composition may be administered for any length of time, and if necessary may be administered as long as the symptoms, disease, or disorder remains in the subject. Dosages may also be varied during the course of treatment. For example, the dosages may be adjusted if the subject encounters side effects, develops unrelated complications, and/or has a change in the kind, dosage, and/or administration of one or more medications other than those of the combination therapy.

Administration to a subject of the flavokine therapy may start before, during, or after symptoms or evidence of viral infection appear. In one embodiment, treatment is started as early as immediately, 15 minutes (min)., 30 min., 1 hour(s) (hr.), 1½ hr., 2 hr., 2½ hr., 3 hr., 4 hr., 5 hr., 6 hr., 7 hr., 8 hr., 9 hr., hr., 11 hr., 12 hr., 16 hr., 18 hr., 20 hr., 22 hr., 24 hr., 36 hr., 48 hr., 60 hr., 72 hr., 84 hr., 96, hr., 5 days, 6, days, 10 days, 13 days, 1 week, 2 weeks, three weeks, 4 weeks, 6 weeks, 8 weeks, 10 weeks, 12 weeks, 1 month, 2 months, 3, 4 months, 5 months, 6 months, 7 months, 8 months, 9 months, 10 months, 11 months, 12 months, 13 months, 14 months, 15 months, 16 months, 17 months, 18 months, or more, or any intermediate length of time, following evidence of a viral infection or episode. In some embodiments, the therapy is continued for, hours, days (e.g., 1, 2, 3, 4, 5, 6, 7, 8, 9, 10, or more), 1-10 weeks, 1-12 months, or years, or whenever a persistent virus or its symptoms reappear.

A flavokine-based composition may be administered continuously, every 15 minutes 30 min., 1 hour(s) (hr.), 1½ hr., 2 hr., 2½ hr., 3 hr., 4 hr., 6 hr., 8 hr., 12 hr., 24 hr., 36 hr., 48 hr., 3 days, 4 days, 5 days, 6, days, 1 week, 2 weeks, or frequencies intermediate or less than the foregoing.

In one embodiment dosage is 1-2 drops of the undiluted sap per lesion, twice or more daily with the composition applied to each lesion. Multiple drops are applied to a crop of lesions. The drops are allowed to dry (several minutes) or they are gently rubbed (about 15 seconds) over the lesions until the composition changes to a “creamier” state. It then dries very quickly (several seconds). Rubbing should be gentle to prevent autoinoculation of uninfected tissue.

In one embodiment, a flavokine-based composition is first applied to a bandage (e.g., gauze), which is then applied to the lesion. This means of application is particularly useful for difficult regions of the human body (groin, armpit, and eyes). The treated bandage is applied to each lesion. If the bandage is separated from the lesion or if the dressing has been worn for 24 hours, a new, treated bandage should be applied. A new dressing is generally applied every day. In one embodiment, the composition is administered until the symptoms (e.g., skin lesions) disappear, become less pronounced, or problematic side effects occur.

Materials and methods of the invention can be practiced on animals, to treat animal viral infections and other skin conditions caused by Poxviridae. Treatment of any domestic pet animal, livestock, zoo animals, circus animals, endangered species, and the like is contemplated.

Flavokine-based compositions are administered in an amount effective to palliate the symptoms of the conditions and symptoms caused by Poxviridae. As indicated herein, any form of administration and pharmaceutical composition is contemplated. Those of ordinary skill in the art will readily optimize effective dosages and administration regimens as determined by good medical practice and the clinical condition of the individual subject, taking into account such considerations as therapeutic efficacy, risk of toxicity, and side-effects.

Thus it will be appreciated that a method of treatment using flavokine in accord with the invention, for treating the Poxviridae family of infections and their associated symptoms such as but not limited to viral induced lesions, is comprised of identifying a subject infected with a virion found in the Poxviridae family and administrating a therapeutically effective amount of flavokine including synthesized analogs thereof.

The invention further encompasses compounds and methods for treating subjects, infected with any member of the Poxviridae family, including subfamilies and genera discovered, undiscovered, presumed eradicated, created, mutated, or yet to evolve or exist. This invention includes Poxviridae native to humans and Poxviridae native to animals but found in human for the purpose of biologic terrorism and those that are not.

In one embodiment, the invention is prophylactic treatment for any of the viral infections described herein, comprising (a) selecting a subject in need of prophylaxis for the Poxviridae family virions, e.g., by identifying or diagnosing the presence of a viral infection in a subject or identifying a risk for infection due to infected members of the subject's family, community, etc; and (b) administering to the subject a composition wherein the composition is flavokine or synthesized analogs thereof. Subjects include humans, zoo mammals, mammals domesticated as pets, livestock, and racing animals, including but not limited to felines, bovines, canines, equines, porcines, dromedaries, and others; and birds, including but not limited to zoo birds, pets, and farm birds, e.g., eagles, hawks, canaries, parrots, chickens, turkeys, ostrich, and emu.

In one embodiment, a flavokine composition may be arranged in a kit or package by unit dose, to permit co-administration with one or more other therapeutic agents, but the flavokine-based composition and the agent are not in admixture. The term “unit dose” as used herein refers to a quantity sufficient to deliver one treatment to a subject identified as having a viral infection or identified as at risk for infection with the Poxvirida family of viruses.

In another embodiment, a flavokine composition may be arranged in a kit or package by unit dose, to permit co-administration with one or more other therapeutic agents and the flavokine composition and the agent are in admixture. In another embodiment, components to the kit, package by unit dose are packaged with instructions for administering the agents to a subject for treatment. In another embodiment, stabilizers which are known in the art are added so that the composition is storage stable at least about 12 months.

As used herein “treating” refers to preventing, curing, and/or ameliorating an infection and/or the symptoms associated with or induced by the Poxviridae family of viral infections including but not limited to the reduction of the lesion number and/or size, and/or duration of lesions on the skin, scalp, mouth, nasal cavity, genitals, and other surfaces and/or duration of the infection.

As used herein, the term “antiviral activity” refers to the ability of the composition, method, or treatment regimen to reduce the size, extent, severity, and duration of infections, lesions, or the communicability caused by or associated with the Poxviridae family of viruses.

As used herein, the term “administration” refers to the process whereby the composition or method of the invention is introduced to a human or animal, which is the host of a virus in the Poxviridae family, and is in need of treatment for infection.

As used herein, the term “therapeutically effective” refers to when a composition or method of the invention is properly administered in vivo to a subject and a measurable beneficial effect occurs. Exemplary beneficial effects are described throughout the application, and include measurable antiviral effects in conditions where viral load can be assayed; a reduction of clinically verifiable and/or patient-reported symptoms, including the reduction, impedance or retardation in the growth of lesions; shrinkage of lesions; reduction in the duration of the symptoms caused by the Poxviridae family directly or indirectly; or complete resolution or curing of the viral infection or outbreak.

In one embodiment, it is contemplated that flavokine or other compositions of the invention are administered to patients in need of treatment in combination with other therapeutics, such as a second agent which is an antiviral agent. When given in combination with another agent, the amount of flavokine given may be reduced accordingly. Second agents are administered in an amount determined to be safe and effective at ameliorating human disease.

It is contemplated that the antiviral agents are administered in the same formulation as flavokine and given simultaneously. Alternatively, the agents may be administered in a separate formulation and still be administered concurrently with flavokine. As used herein, concurrently refers to agents given within 30 minutes of each other. The second agent may also be administered prior to administration of flavokine. Prior administration refers to administration of the agent within the range of one week prior to flavokine treatment up to 30 minutes before administration of flavokine. It is further contemplated that the second agent is administered subsequent to administration of the flavokine composition. Subsequent administration is meant to describe administration from 30 minutes after flavokine administration up to one week after flavokine treatment.

Novel formulations that include flavokine and a second therapeutic agent are themselves aspects of the invention. Such dual agent formulations or kits, when packaged together but not in admixture, may include pharmaceutically acceptable diluents, carriers, stabilizers, or the like, or delivery agents.

The foregoing description of the invention is not intended to be limiting or comprehensive, and additional embodiments are contemplated. All such embodiments are aspects of the invention. Moreover, for the sake of brevity, various details that are applicable to multiple embodiments have not been repeated for every embodiment. Variations reflecting combinations and rearrangements of the embodiments described herein are intended as aspects of the invention. In addition to the foregoing, the invention includes, as an additional aspect, all embodiments of the invention narrower in scope in any way than the variations specifically mentioned above. For example, for aspects described as a genus or range, such as dosages or dosing regimens, every subgenus, sub-range or species is specifically contemplated as an embodiment of the invention.

Without departing from the scope thereof, one skilled in the art can easily ascertain from the forgoing description the essential characteristics of this invention to adapt it to various uses and conditions. The foregoing description and examples have been set forth merely to illustrate the invention and are not intended to be limiting. Because modifications of the disclosed embodiments incorporating the spirit and substance of the invention may occur to persons skilled in the art, the invention should be construed to include everything within the scope of the appended claims and equivalents thereof. The patents, patent application publications and other publications referenced herein are incorporated in their entirety.

Although the applicant invented the full scope of the claims appended hereto, the claims are not intended to encompass within their scope the prior art work of others. Therefore, in the event that statutory prior art within the scope of a claim is brought to the attention of the applicants by a Patent Office or other entity or individual, the applicant reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

What is claimed:

1. A composition comprising: pharmaceutically effective amounts of flavokine for treatment of a viral infection and its associated symptoms wherein the viral infection is selected from the group consisting of cowpox, vaccinia, and combinations thereof.

2. The composition of claim 1, further comprising beta-alanyl-taurine, wherein the concentration of beta-alanyl-taurine is a homeopathic preparation between about 10⁻⁶ and 10^{-100,000} Mol.

3. The composition of claim 2, wherein beta-alanyl-taurine has a homeopathic potency selected from the group of dilutions consisting of 2×, 3×, 4×, 5×, 6×, 7×, 8×, 9×, 10×, 11×, 12×, 15 ×, 20× and 24× HPUS.

4. The composition of claim 2, further comprising an antioxidant.

5. The composition of claim 4, wherein the antioxidant is zinc selected from the group consisting of zinc gluconate, zinc sulfate, and combinations thereof.

6. The composition of claim 5, wherein the concentration of zinc is within the range of about 25-75 mMol.

7. The composition of claim 5, wherein the concentration of zinc is within the range of about 10-100 mMol.

8. The composition of claim 4, wherein the antioxidant is citrus extract selected from the group consisting of orange, lemon, lime, grapefruit, and combinations thereof.

9. The composition of claim 8, wherein the concentration of citrus extract is within the range of about 0.5-5% W/V.

10. The composition of claim 1, further comprising beta-alanyl-taurine wherein the concentration of beta-alanyl-taurine is between about 10⁻⁶ and 10^{-100,000} Mol.

11. The composition of claim 10, further comprising an antioxidant.

12. The composition of claim 11 wherein the antioxidant is zinc selected from the group consisting of zinc gluconate, zinc sulfate, and combinations thereof, further wherein the zinc is within a range of about 10-100 mMol.

13. The composition of claim 11 wherein the antioxidant is citrus extract selected from the group consisting of orange, lemon, lime, grapefruit, and combinations thereof, further wherein the citrus extract is within a range of about 0.5-5% W/V.

14. The composition of claim 1, further comprising a pharmaceutically acceptable carrier.

15. The composition of claim 1, wherein the composition is suitable for topical application.

16. The composition of claim 1, wherein the composition is suitable for ingestion.

17. The composition of claim 1, wherein the composition is suitable for intravenous administration.

18. The composition of claim 1, wherein the composition is storage stable at least about 12 months.

19. A method comprising:
a) Identifying a subject infected with a virion, wherein the virion is from cowpox, vaccinia and combinations thereof; and
b) Administering to the subject a composition comprising: a pharmaceutically effective amount of flavokine for treatment of cowpox, vaccinia and combinations thereof.

20. A method comprising:
a) Identifying a subject infected with a virion wherein the virion is from cowpox, vaccinia and combinations thereof; and
b) Administering to the subject a composition comprising:
i) a pharmaceutically effective amount of flavokine; and
ii) Beta-alanyl-taurine wherein the concentration of beta-alanyl-taurine is between about 10⁻⁶ and 10^{-100,000} Mol; said composition for treatment of cowpox, vaccinia and combinations thereof.

21. A method comprising:
a) Identifying a subject infected with a virion wherein said virion is from cowpox, vaccinia and combinations thereof, and
b) Administering to the subject a composition comprising:
i) a pharmaceutically effective amount of flavokine;
ii) Beta-alanyl-taurine wherein the concentration of beta-alanyl-taurine is between about 10⁻⁶ and 10^{-100,000} Mol; and
iii) an antioxidant; said composition for treatment of cowpox, vaccinia and combinations thereof.

22. The method of claim 21 wherein the antioxidant is zinc selected from the group consisting of zinc gluconate, zinc sulfate, and combinations thereof, further wherein the zinc within a range of about 10-100 mMol.

23. The method of claim 21 wherein the antioxidant is citrus extract selected from the group consisting of orange, lemon, lime, grapefruit, and combinations thereof, further wherein the citrus extract within a range of about 0.5-5% W/V.

24. The method of claim 19, wherein the composition is administered topically.

25. The method of claim 19, wherein the composition is administered orally.

26. The method of claim 19, wherein the composition is administered intravenously.

27. A kit comprising:
a) At least one unit dose of pharmaceutically effective amounts of flavokine; and
b) Beta-alanyl-taurine wherein the concentration of beta-alanyl-taurine is between about 10⁻⁶ and 10^{-100,000} Mol; said kit for treatment of a viral infection and its associated symptoms wherein the viral infection is selected from the group consisting of cowpox, vaccinia and combinations thereof.

28. A kit comprising:
a) At least one unit dose of pharmaceutically effective amounts of flavokine;
b) Beta-alanyl-taurine wherein the concentration of beta-alanyl-taurine is between about 10⁻⁶ and 10^{-100,000} Mol; and
c) An antioxidant; said kit for treatment of a viral infection and its associated symptoms wherein the viral infection is selected from the group consisting of cowpox, vaccinia and combinations thereof.

29. The kit of claim 28 wherein the antioxidant is zinc selected from the group consisting of zinc gluconate, zinc sulfate and combinations thereof, further wherein the zinc within a range of about 10-100 mMol.

30. The kit of claim 28 wherein the antioxidant citrus extract selected from the group consisting of orange, lemon, lime, grapefruit and combinations thereof, further wherein the citrus extract within a range of about 0.5-5% W/V.
